(19) United States
(12) Patent Application Publication
(10) Pub. No.: US 2022/0411849 A1
(43) Pub. Date: Dec. 29, 2022

(54) METHOD FOR ISOLATING NUCLEIC ACIDS

(71) Applicant: NASASBIOTECH, S.L., A Coruña (ES)

(72) Inventors: Alexandre DE LA FUENTE GONZÁLEZ, La Coruña (ES); Miguel ABAL POSADA, A Coruña (ES); Laura MUINELO ROMAY, Milladoiro, Ames, A Coruña (ES); Carlos CASAS AROZAMENA, Pontevedra (ES)

(21) Appl. No.: 17/773,895

(22) PCT Filed: Nov. 4, 2020
(86) PCT No.: PCT/EP2020/080933

§ 371 Date: May 3, 2022

(30) Foreign Application Priority Data
Nov. 4, 2019 (EP) 19382960.3

(51) Int. Cl. C12Q 1/6806

(52) U.S. Cl. CPC C12Q 1/6806; C12Q 1/686

(57) ABSTRACT

The invention relates to an in vitro method for isolating nucleic acids associated to or contained inside extracellular vesicles (EVs) from a sample based on the formation of a DMB-EVs precipitate and the isolation of the nucleic acids present in the precipitate. The invention also relates to the use of the method of the invention for diagnosing or for determining the susceptibility of a subject to a disease, for determining the prognosis or for monitoring the progression of a disease, for monitoring the effect of a therapy, for identifying compounds suitable for the treatment of a disease, or for designing a personalized therapy or selecting a patient susceptible to being treated with a therapy for the prevention and/or treatment of a disease. In addition, the invention also relates to a kit comprising dimethylmethylene blue (DMB) and a reagent capable of isolating nucleic acids from EVs, and to its use.

### FIELD OF THE INVENTION

The present invention relates to the field of methods for isolating nucleic acids from a sample and to diagnostic methods.

### BACKGROUND OF THE INVENTION

Cancer remains one of the leading causes of morbidity and mortality in the world. The development of specific medicine, with the aim of tailoring therapies for patients in relation to the personalized patterns of the tumor, it is expected to improve diagnosis and treatment, reducing the morbidity and mortality rate and the indirect costs associated with cancer.

The knowledge of the biology and genetics of tumors improves the decisions to be made regarding treatment, which can be adapted to the specific characteristics of each patient in a personalized medicine. In fact, many studies have established that the genomic landscape of tumors and metastases dynamically evolve over time in response to selective pressure of therapies that can suppress or promote the growth of different cellular clones. Tissue biopsy provides a tumor picture limited to a single time point, is invasive, charged with potential complications, cannot be obtained when clinical conditions have worsened or when a tumor is inaccessible and may also show the genetic heterogeneity of numerous tumor subclones. These limitations are particularly evident in the presence of acquired resistance to therapy or in monitoring the disease during follow up.

Liquid biopsy—based both on the analysis of circulating tumor cells (CTC), of circulating tumor DNA, and of biomarkers present in blood components, such as extracellular vesicles (EVs)—allows to study how the oncological disease evolves through a minimally invasive sample. Extracellular vesicles and their nucleic acids have been proposed for the development of EV-based biomarkers and personalized medicine (Fatemeh Momen-Heravi et al., Pharmacology & Therapeutics Volume 192, December 2018, Pages 170-187).

An important technical factor that needs to be considered when selecting EVs isolation protocols across different biofluids is the volume of starting material, as some biofluids may need to be concentrated prior to EVs isolation. A limitation of most of these techniques is the efficiency in the recovery of sufficient amounts of EVs starting from small volumes of biological samples. In addition, EVs purification methods based on differential ultracentrifugation or the density gradient ultracentrifugationre influenced by several parameters which are difficult to standardize such as the viscosity of biofluids. In addition, the integrity of EVs after prolonged high speed ultracentrifugation may be damaged. In fact, membrane debris are often observed by electron microscopy and the recovery of exosomal RNA and proteins is not optimal. On the other hand, size exclusion chromatography does not guarantee the removal of several small contaminants, does not avoid the loss of EVs by binding to membranes and may cause deformation of vesicles.

Therefore, alternative methods for isolating nucleic acids associated to or contained inside EVs are needed in the art.

### SUMMARY OF THE INVENTION

Therefore, in a first aspect, the invention relates to an in vitro method for isolating nucleic acids associated to or contained inside extracellular vesicles (EVs) from a sample which comprises:
a) contacting the sample with the dimethylmethylene blue (DMB) dye at a pH comprised between 2 and 6.9;
b) incubating the mixture from a) at a temperature comprised between 0° C. and 40° C. for the time required for the formation of a DMB-EVs precipitate;
c) recovering the DMB-EVs precipitate; and
d) isolating the nucleic acids present in the precipitate.

In a second aspect, the invention relates to the use of the method of the invention for diagnosing a disease or for determining the susceptibility of a subject to a disease.

In a third aspect, the invention relates to the use of the method of the invention for determining the prognosis or for monitoring the progression of a disease in a subject.

In a fourth aspect, the invention relates to the use of the method of the invention for monitoring the effect of a therapy for the treatment of a disease.

In a fifth aspect, the invention relates to the use of the method according the invention for identifying compounds suitable for the treatment of a disease. In a sixth aspect, the invention relates to the use of the method of the invention for designing a personalized therapy in a subject or for selecting a patient susceptible to being treated with a therapy for the prevention and/or treatment of a disease.

In a seventh aspect, the invention relates to a kit comprising dimethylmethylene blue (DMB) and a reagent capable of isolating nucleic acids from EVs.

In an eight aspect, the invention relates to the use of a kit comprising DMB or the kit according to the invention for isolating nucleic acids associated to or contained inside EVs.

### BRIEF DESCRIPTION OF THE DRAWINGS

FIG. 1: Graphic description of the method of EVs isolation by using DMB and the possibilities of analysis by different approaches that are compatible with the technique and that allow the analysis of the genetic material contained and associated to the isolated EVs.

FIG. 2: NTA nanosight NS300 particle tracking profile of plasma EVs isolated by DMB and ultracentrifugation. (A) Representative image of video recorder for the EVs particles from plasma isolated by DMB (upper panel) and ultracentrifugation (lower panel). (B) Representative image of EVs isolated by DMB (upper panel) and ultracentrifugation (lower panel), expressed as particles size (nm) and concentration (particles/ml).

FIG. 3: Exosome characterization by western-blot.

FIG. 4: Visualization of isolated EVs by scanning and transmission electron microscopy (TEM). (A) urine sample; (B), plasma sample; (C) and (D) plasma sample incubated with anti-CD9 antibody.

FIG. 5: Efficiency (A) and purity (B) of EVs from culture media isolated by DMB and ultracentrifugation (ultra).

FIG. 6: Purity analysis of isolated EVs by different methods.

FIG. 7: Extraction and quantification of DNA associated to EVs isolated by DMB (EXOGAG) and cell-free DNA (cfDNA).

FIG. 8: Levels of point mutations identified by ddPCR using DNA from plasmatic EVs isolated with DMB (EXOGAG) and also cell-free DNA (cfDNA). MAFS, mutant allele fraction.

FIG. 9: Levels of point mutations identified by ddPCR and BEAMing using DNA from plasmatic EVs isolated with DMB (EXOGAG) and also cell-free DNA (cfDNA). MAFS, mutant allele fraction.

FIG. 10: Evaluation by nano-tracking analytical particle (NTA) technology of isolated EVs from saliva.

FIG. 11: RNA and microRNA quantification from saliva EVs of three different samples (A, B, C) using the DMB-based precipitation technique. FU: fluorescence; nt: nucleotide size.

FIG. 12: miRNA expression analysis by RT-qPCR assay in EVs isolated by DMB. Relative miR expression normalized to cel-miR-39.

FIG. 13: Mean Quality Scores of WES analysis on EVs-DNA from plasma. The y-axis on the graph shows the quality scores. The higher the score, the better the base call. The background of the graph divides they axis into very good quality calls (upper zone), calls of reasonable quality (medium zone), and calls of poor quality (lower zone). The three samples analyzed in the study showed good quality scores.

FIG. 14: MSI (A) and CNV (B) analysis in plasma EVs isolated by DMB (evDNA) and also cell-free DNA (cfDNA).

FIG. 15. Methylation analysis in culture medium (A) and plasma (B) EVs isolated by DMB (evDNA) and also genomic DNA (gDNA) and cell-free DNA (cfDNA).

FIG. 16. mRNA analysis in plasma (A) and urine (B) EVs purified by DMB.

### DETAILED DESCRIPTION OF THE INVENTION

The inventors have observed that dimethylmethylene blue dye (DMB), at an acidic pH, is suitable for isolating nucleic acids associated to or contained inside extracellular vesicles (EVs) from a sample. Therefore, a new method for isolating nucleic acids in an easy and efficient way has been developed. The genetic content of extracellular vesicles can be used for diagnosis, prognosis or monitoring of pathologies; and for the development and identification of new therapeutic targets in oncological, rheumatic, degenerative, renal diseases, or any pathology where damaged tissues have the capacity to generate and secrete EVs.

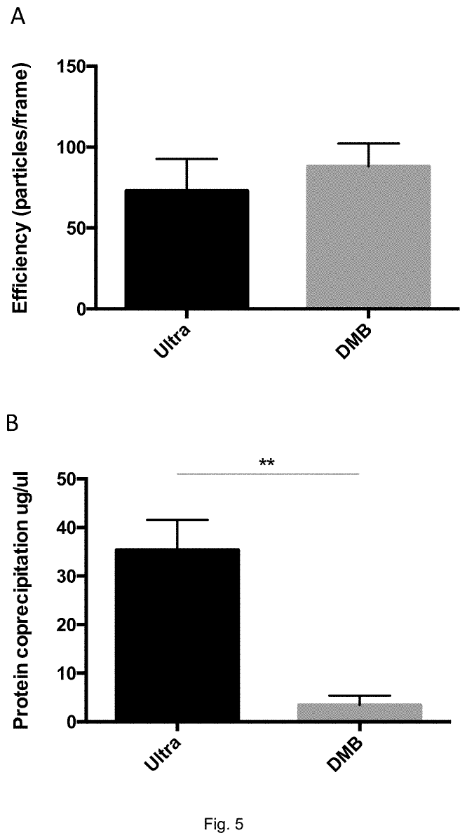

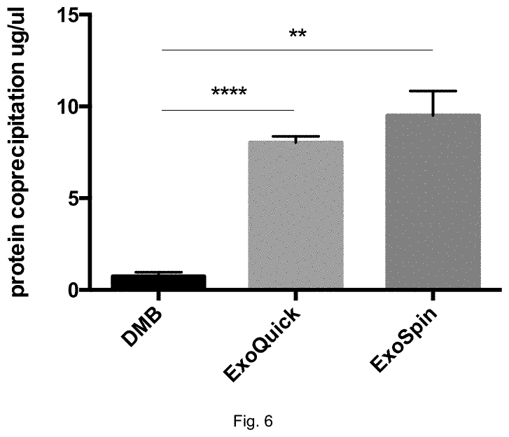

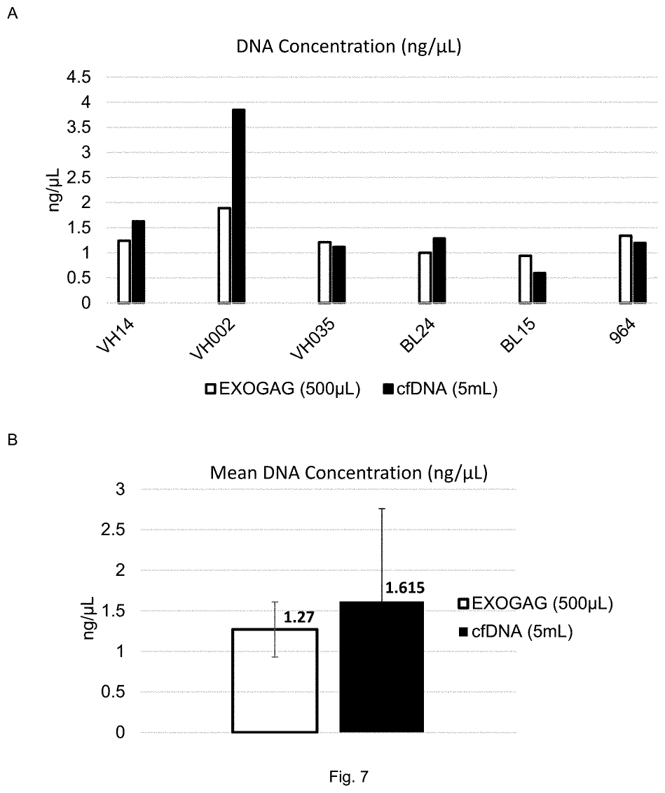

The method of the invention requires a small quantity of sample (0.5 ml) to isolate enough extracellular vesicles to obtain the amount of genetic material equivalent to that obtained from 5 ml of sample when other methods of the prior art are used (Example 7 and FIG. 7). Furthermore, the method of the invention allows precipitating the extracellular vesicles of a sample without the need of a previous step of enrichment or without isolating previously the extracellular vesicles from the sample. Additionally, the method of the invention reduces the levels of co-precipitated contaminating proteins obtained when compared with the methods of the prior art (FIGS. 5B and 6).

Thus, in a first aspect, the invention relates to an in vitro method for isolating nucleic acids associated to or contained inside extracellular vesicles (EVs) from a sample which comprises:
a) contacting the sample with the dimethylmethylene blue (DMB) dye at a pH comprised between 2 and 6.9;
b) incubating the mixture from a) at a temperature comprised between 0° C. and 40° C. for the time required for the formation of a DMB-EVs precipitate;
c) recovering the DMB-EVs precipitate; and
d) isolating the nucleic acids present in the precipitate.

“Isolating nucleic acid”, as used herein, relates to the act or action to separate or purify nucleic acids from a sample to allow subsequent analyses such as PCR based analyses.

As used herein, the term “nucleic acids” means either or both of deoxyribonucleic acids (DNA) and ribonucleic acids (RNA). Isolated nucleic acids may comprise single type of nucleic acids or 2 or more different types of nucleic acids. They may be single-stranded, double-stranded, linear or cyclic. Length of isolated nucleic acids is also not limited. Length of nucleic acids isolated by the present invention may be in a range of from about 1 bp to about 1,500 kbp, preferably of from about 1 kbp to about 500 kbp, more preferably from about 20 kbp to about 200 kbp. In a preferred embodiment, the length of the nucleic acids isolated by the present invention are about 25 bp, 149 bp, 155 bp, 680 bp, 1500 bp. In a more preferred embodiment, the average size of the nucleic acid obtained was around 150 bp. Illustrative, non-limitative examples of nucleic acids that can be isolated according to the method of the invention are miRNA transfer RNAs (tRNAs), ribosomal RNAs (rRNAs), microRNAs, siRNAs, piRNAs, snoRNAs, snRNAs, exRNAs, scaRNAs, lncRNAs.

The term nucleic acids includes modified nucleic acids and conjugated nucleic acids. The term “nucleic acid” also refers to molecules formed by non-conventional nucleotides bound as well as variants thereof, including modifications in the purine or pyrimidine residues and modifications in the ribose or deoxyribose residues. Examples of modified nucleotides that can be used in the present invention include, but are not limited to, nucleotides having at position 2′ of the sugar a substituent selected from the fluoro, hydroxyl, amino, azido, alkyl, alkoxy, alkoxyalkyl, methyl, ethyl, propyl, butyl group or a functionalized alkyl group such as ethylamino, propylamino and butylamino. Alternatively, the alkoxy group is methoxy, ethoxy, propoxy or a functionalized alkoxy group according to the formula —O(CH2)q-R, where q is 2 to 4 and R is an amino, methoxy or ethoxy.

In a preferred embodiment of the method of the invention, the nucleic acid is DNA. In another preferred embodiment of the method of the invention, the nucleic acid is RNA.

“Associated to or contained inside extracellular vesicles (EVs)”, related to the nucleic acids, means that the nucleic acids are inside the EVs or associated to the membrane of the vesicles.

“Extracellular vesicles (EVs)”, as used herein, relates to a heterogeneous vesicle populations spanning 50 to 10,000 nm in size (smaller than a biological cell) surrounded by a membrane which originated from a biological cell. This sphere varies greatly depending on the origins of the cells in which it is made or the way it is made. In this invention, the EVs include any one selected from the group consisting of exosomes, ectosomes, microvesicles, oncosomes, microparticles, dexosomes, texosomes and apoptotic bodies, and preferably are exosomes. Extracellular vesicles are membrane enclosed vesicles released by all cells. Based on the biogenesis pathway different types of vesicles can be identified: (1) Exosomes are formed by inward budding of late endosomes forming multivesicular bodies (MVB) which then fuse with the limiting membrane of the cell concomitantly releasing the EVs. (2) Microvesicles or shedding vesicles are formed by outward budding of the limiting cell membrane followed by fission. Finally, (3) when a cell is dying via apoptosis, the cell is disintegrated and divides its cellular content in different membrane enclosed vesicles termed apoptotic bodies. These mechanisms allow the cell to discard waste material and were more recently also associated with intercellular communication. Their primary constituents are lipids, proteins and nucleic acids. They are composed of a protein-lipid bilayer encapsulating an aqueous core comprising nucleic acids and soluble proteins. Molecular markers such as CD63, CD81 and Annexin V are used to classify EVs.

In a preferred embodiment the EVs are exosomes. As it is used herein, the term “exosomes” refers to small extracellular nanovesicles (50-200 nm) surrounded by a membrane, said nanovesicles originating from the endocytic pathway and being released by different cell types into most biological fluids, including urine. They are also secreted by cells in vitro. Their functions include, among others, intercellular RNA and membrane receptor traffic, induction of immunity and antigen presentation, modulation of bone mineralization, and anti-apoptotic responses. Their membranes are rich in proteins involved in transport and fusion, as well as lipids such as cholesterol, sphingolipids, ceramides, etc.

Exosomes are identified because they show a range of density between 1.13 and 1.19 g/ml when separated in a sucrose gradient, and in that they possess a series of markers such as CD63, CD81, CD9, ALIX, FLOT1, ICAM1, EpCAM, ANXAS, TSG101, and Hsp70 which can be detected, for example, by means of antibodies. In a preferred embodiment, the exosomes have a diameter of 100 to 170 nm, more preferably 100 to 150 nm, even more preferably 150 nm. Other markers of exosomes are Tetraspanins (CD61, CD 81, CD82, CD9), ESCRT components, TSG101, Flotillin 1 and Flotillin 2, HSPs, ALIX, MFGE8. In a preferred embodiment, exosomes are identified by CD9 marker.

In another preferred embodiment, the EVs are microvesicles. The term “microvesicles”, also called shedding vesicles, shedding microvesicles, or microparticles refers to EVs of approximately 100-1000 nm in diameter and originate from the outward budding of the plasma membrane. Microvesicles are characterized by the surface markers Annexin V, Integrins and CD40 ligand.

In another preferred embodiment, the EVs are apoptotic bodies. Apoptotic vesicles are a subpopulation of EVs that range from 100-2000 nm in diameter and are generated by the blebbing of plasma membrane of cells undergoing apoptosis. Apoptotic bodies are characterized by the surface marker Annexin V, particularly enriched in phosphatidylserine.

In another preferred embodiment, the EVs of the invention do not contain exosomes.

In another preferred embodiment, the EVs are GAG-EVs. As it is used herein, the term “glycosaminoglycan” or “GAG”, also called mucopolysaccharide, refers to a heteropolysaccharide formed by repetitions of disaccharide units. Glycosaminoglycans are linear chains in which β1→3 bonds alternate with β1→4 bonds of a uronic acid (D-glucuronic or L-iduronic) bound by means of a β1→3 bond to an amino sugar (N-acetyl-glucosamine or N-acetylgalactosamine). GAGs are differentiated according to the nature of the disaccharide units forming them, the length of the disaccharide chain (10-150 units), and the modifications thereof (N-sulfation, 0-sulfation, N-acetylation, or epimerization of the saccharide units). The following seven GAGs stand out among those of biological interest: hyaluronic acid (HA), chondroitin-4-sulfate (C4S), chondroitin-6-sulfate (C6S), dermatan sulfate (DS) or chondroitin sulfate B, keratan sulfate (KS), heparan sulfate (HS), and heparin (HEP). They have a high density of negative electrical charge due to the introduction of acidic groups (carboxy, esterified sulfates, and sulfamide) in their structure. They undergo variable degrees of sulfation, where the sulfate esterified to alcoholic OH increases their polyanionic character. The number of negative charges per disaccharide unit varies between 1, in the case of hyaluronic acid and keratan sulfate, and 4 in the case of heparin. GAGs are associated to EVs. In an embodiment, the EVs are GAG-exosomes. In another embodiment, the EVs are sulfated GAG-EVs.

In the context of the invention, the term “sample” refers to any type of sample which contains or is susceptible of containing nucleic acids associated to or inside EVs. In a preferred embodiment, the sample is a biological sample.

As it is used herein, the term “biological sample” refers to any material originating from human beings, animals, or plants which can contain information relating to their genetic endowment. Examples of biological samples that can be used in the present invention are, without limitation, samples of urine, serum, plasma, saliva, tissues, cells, EVs, synovial fluid, vitreous humor, cerebrospinal fluid, skin, intestinal mucosa, peritoneal fluid, arterial wall, bone, cartilage, embryonic tissue, and umbilical cord, etc. In a preferred embodiment the sample is liquid biopsy, preferably serum, plasma, urine, saliva, synovial fluid, ascitic fluid, cerebrospinal fluid, or semen; more preferably the liquid biopsy is selected from the group consisting of plasma, urine, ascitic fluid and saliva. A liquid biopsy, also known as a fluid biopsy or fluid phase biopsy refers to non-solid biological samples, preferably blood. In another preferred embodiment the sample is a tissue sample.

A suitable sample volume of a bodily fluid is, for example, in the range of about 0.05 ml to about 30 ml fluid. The volume of fluid may depend on a few factors, e.g., the type of fluid used. For example, the volume of serum or plasma samples may be about 0.05 ml to about 0.5 ml, preferably about 0.1 ml to 5 ml. The volume of urine samples may be about 1 ml to about 30 ml, preferably about 10 ml.

The method of the invention comprises in a first step contacting the sample with the dimethylmethylene blue (DMB) dye at a pH comprised between 2 and 6.9. This contacting involves mixing the sample and DMB until obtaining a homogeneous mixture. The sample can be mixed, without limitation, by inverting the tube or vortexing.

As it is used herein, the term “dimethylmethylene blue” or “DMB” refers to a cationic dye, also known as 1,9-dimethylmethylene blue, comprising the compound 3,7-bis-(dimethylamino)-1,9-dimethyldiphenothiazin-5-ium and any salt thereof. The salts thereof include, among others, salts with anions derived from inorganic acids, for example and without limitation, hydrochloric acid, sulfuric acid, phosphoric acid, diphosphoric acid, bromic acid, iodide, nitric acid, and organic acids, for example and without limitation, citric acid, fumaric acid, maleic acid, malic acid, mandelic acid, ascorbic acid, oxalic acid, succinic acid, tartaric acid, benzoic acid, acetic acid, methanesulfonic acid, ethanesulfonic acid, benzenesulfonic acid, cyclamic acid, or p-toluenesulfonic acid. In a preferred embodiment, the DMB is 3,7-bis-(dimethylamino)-1,9-dimethyldiphenothiazin-5-ium chloride. The term DMB also includes mixed salts. In a preferred embodiment, DMB is a double 3,7-bis-(dimethylamino)-1,9-dimethyldiphenothiazin-5-ium zinc chloride salt. These compounds can be acquired commercially.

DMB is a powder substance which is dissolved in a suitable solvent, such as for example, ethanol, until reaching a suitable concentration. In a preferred embodiment, DMB is at a concentration ranging between 0.01 and 100 mM, preferably between 0.29 and 0.35 mM, more preferably at 0.29 mM or 0.30 mM, more preferably 0.29 mM. In a preferred embodiment, the solvent in which the dye is dissolved is ethanol.

DMB used in the method of the invention must be at an acidic pH comprised between 2 and 6.9.

The term “pH” refers to the measurement of the acidity or alkalinity of a solution. pH typically ranges from 0 to 14 in an aqueous solution, where solutions with pH below 7 are acidic and solutions having pH above 7 are alkaline. pH=7 indicates the neutrality of the solution, where the solvent is water. The pH of a solution can be precisely determined by means of a potentiometer (or pH-meter), and it can also estimated by means of indicators, by methods that are well known in the state of the art. Given that the pH value may vary with temperature, in the context of this invention the pH is measured at 20° C. DMB used in the first method of the invention has a pH measured at 20° C. comprised between 2 and 6.9; preferably comprised between 3 and 4; more preferably comprised between 3.5 and 4; more preferably comprised between 3.3 and 3.6. In a preferred embodiment, the pH measured at 20° C. is 3.5.

For the DMB dissolved in a suitable solvent to have an acidic pH, a buffer agent must be added. In the context of the present invention, “buffer agent” is understood as an agent capable of controlling the acidic pH of the solution and keeping it constant at a pH comprised between 2 and 6.9. Buffer agents suitable for the present invention are, without limitation, acetate buffer, phosphate citrate buffer, diphosphate buffer, formiate buffer, and a combination thereof or reagents as glycine or sodium chloride. In a preferred embodiment of the invention, the buffer agent is sodium formiate, preferably 0.2 M sodium formiate at pH 3.5. In a preferred embodiment, the buffer agent is mixed with DMB previously dissolved in a suitable solvent such as ethanol, in a DMB dissolved/buffer ratio of 1/99 to 10/90. Preferably, the DMB dissolved/buffer ratio is 1/99.

To analyze the sample, it must be mixed with buffered DMB in a suitable sample:buffered DMB ratio (v/v) so that saturation occurs, such as the ratio comprised in the interval of 1:0.5 to 1:10 (v/v), preferably 1:0.5 to 1:5 (v/v), more preferably 1:1 to 1:5 (v/v). Preferably, they are mixed in a ratio of 1:2 (v/v).

In a preferred embodiment of the method of the invention, step a) is performed without previously isolating EVS from the sample.

Before step a) the sample may be centrifuged to remove large unwanted particles, cells, and/or cell debris, the samples may be centrifuged at a low speed of about 100-500 g, preferably about 250-300 g. Samples can also be centrifuged at a speed between 2,000 g and 10,000 g. As a way of illustrative non-limitative example, the centrifugation may be performed at 2000 g during 10 minutes. In a preferred embodiment, before step a), the centrifugation step to remove unwanted particles, cells, and/or cell debris does not isolate EVs. A skilled person in the art knows methods for isolating EVs which are not applied before step a) in the method of the invention, for example by centrifugation at 100,000 g. The centrifugation step or steps may be carried out at below-ambient temperatures, for example at about 0-10° C., preferably about 1-5° C., e.g., about 3° C. or about 4° C.

The method of the invention comprises in a second step incubating the mixture from a) at a temperature comprised between 0° C. and 40° C. for the time required for the formation of a DMB-EVs precipitate. Incubation can be performed at a temperature comprised between 0° C. and 40° C., preferably between 4° C. and 30° C., more preferably between 10° C. and 28° C., even more preferably between 15° C. and 25° C., still more preferably between 20° C. and 25° C. In a preferred embodiment, the incubation of step b) is performed at 4° C. Incubation will be performed in a cold environment, in a temperate environment, or in an oven depending on the temperature to be reached using methods known to one skilled in the art. In a preferred embodiment, incubation is performed at room temperature (between 20° C. and 25° C.).

In the context of the first method of the invention, “precipitate” is understood as the insoluble solid that is produced by the complex formed between the GAGs, preferably the sulfated GAGs present in the sample to be analyzed, and DMB. In most cases, the precipitate drops to the bottom of the solution and its formation can be seen with the naked eye. In other cases, the precipitate can float or remain in suspension, depending on if it is less dense than or as dense as the rest of the solution.

The incubation time is the time required for the formation of the precipitate and can be determined by one skilled in the art by simple observation of the solution or by methods known in the state of the art. Once the precipitate is formed, it can remain unchanged for days in a temperature range comprised between 0° C. and 40° C. In a preferred embodiment, the incubation time is comprised between 1 minute and 2 hours, where it is preferably at least 1 minute, at least 2 minutes, at least 5 minutes, at least 10 minutes, at least 15 minutes, at least 20 minutes, at least 30 minutes, at least 40 minutes, at least 50 minutes, at least 60 minutes, at least 90 minutes. In a more preferred embodiment, the time required for the formation of the precipitate is at least 5 minutes, more preferably at least 15 minutes. In a preferably embodiment, the incubation is performed during 5 minutes, preferably at 4° C. 5 minutes.

Step c) of the method of the invention comprises recovering the DMB-EVs precipitate. This recovery may consist of obtaining the isolated precipitate separately from the supernatant after step c). Optionally, the precipitate obtained after step c) can be dissolved or mixed with a suitable solvent or solution depending on the subsequent use to be made of the precipitate.

In a preferred embodiment, step c) is performed by centrifugation. The term “centrifugation”, as used herein, refers to subjecting the DMB-EVs precipitated to a centrifuge force in order to separate said precipitate based on their different behaviour upon exerting said centrifugal force. The “speed sufficient to precipitate DMB-EVs” can be determined by the skilled person depending on the size of the EVs. In a particular embodiment, the speed sufficient to precipitate EVs is between 4.000 g and 20.000 g, more preferably 16.000 g. In a particular embodiment, the centrifugation lasts between 1 minute and 1 hour. In a more particular embodiment, the centrifugation lasts between 2 minutes and 30 minutes, preferably between 5 minutes and 15 minutes, more preferably lasts 5 minutes. In an even more particular embodiment the centrifugation lasts about 15 minutes. Illustrative, non-limitative, the centrifugation can be performed at 3000-20000 g, for example at 16.000×g 15 minutes. Illustrative, non-limitative examples of combination of tine and g are shown in Table 1. The centrifugation temperature can be the same as the incubation temperature. All the embodiments related to the incubation temperature are applicable here. In a preferred embodiment, the centrifugation temperature is 4° C. After centrifugation, the supernatant is removed and the pellet contains the EVs. Optionally, this pellet can be resuspended in an appropriate buffer depending on the subsequent use of the EVs material.

In addition, the method of the invention comprises a fourth step, step d), which comprises the isolation of the nucleic acids present in the precipitate. Several methods can be used to isolate nucleic acids from a sample, in particular from EVs.

Following the isolation of EVs from a biological sample, nucleic acids may be extracted from the isolated or enriched EVs fraction. To achieve this, the EVs may first be lysed. The lysis of EVs and extraction of nucleic acids may be achieved with various methods known in the art. The nucleic acid extraction may be achieved using phenol:chloroform according to standard procedures and techniques known in the art or any other lysis buffer. Such methods may also utilize a nucleic acid-binding column to capture the nucleic acids contained within the EVs. Once bound, the nucleic acids can then be eluted using a buffer or solution suitable to disrupt the interaction between the nucleic acids and the binding column, thereby successfully eluting the nucleic acids

The nucleic acid extraction methods may also include the step of removing or mitigating adverse factors that prevent high quality nucleic acid extraction from EVs. Such adverse factors are heterogeneous because different biological samples may contain various species of adverse factors. In some biological samples, factors such as excessive DNA/RNA may affect the quality of nucleic acid extractions from such samples. In other samples, factors such as excessive endogenous RNase may affect the quality of nucleic acid extractions from such samples. Many agents and methods may be used to remove these adverse factors. These methods and agents are referred to collectively herein as an “extraction enhancement operations.” In some instances, the extraction enhancement operation may involve the addition of nucleic acid extraction enhancement agents to the sample. To remove adverse factors such as endogenous DN/RNases, such extraction enhancement agents as defined herein may include, but are not limited to, an RNase inhibitor such as Superase-In (commercially available from Ambion Inc.) or RNaselNplus (commercially available from Promega Corp.), or other agents that function in a similar fashion; a protease (which may function as an RNase inhibitor); DNase; a reducing agent; a decoy substrate such as a synthetic RNA and/or carrier RNA; a soluble receptor that can bind RNase; a small interfering RNA (siRNA); an RNA binding molecule, such as an anti-RNA antibody, a basic protein or a chaperone protein; an RNase denaturing substance, such as a high osmolarity solution, a detergent, or a combination thereof.

The invention also relates to a method for isolating EVs from a sample which comprises:
a) contacting the sample with the dimethylmethylene blue (DMB) dye at a pH comprised between 2 and 6.9;
b) incubating the mixture from a) at a temperature comprised between 0° C. and 40° C. for the time required for the formation of a DMB-EVs precipitate;
c) recovering the DMB-EVs precipitate.

In a preferred embodiment, the EVs are not previously isolated from the sample. In another preferred embodiment, the EVs isolated by the method of the invention do not contain exosomes. Steps a, b and c) of the method for isolating nucleic acids associated to or contained inside extracellular vesicles (EVs) are equally applicable to the steps of this method of the invention, with the appropriate modifications to isolate EVs.

Uses of the Method of the Invention

The nucleic acids obtained by the method of the invention can be subjected to qualitative and quantitative analyses, including sequencing, the determination of the size of DNA and RNA chains, the level of expression of specific DNA or RNA sequences, the number of gene copies, the analysis of different classes of mutations including any alteration of the nucleotide sequence such as substitutions, insertions, or deletions, genomic amplification, rearrangements and microsatellite instability, or any technique that allows analyzing any change or modification that occurs in the genetic material at genomic, transcriptomic and epigenetic level. These analyses can be applied to the field of biomedicine since this information can be used to detect, predict or monitor different pathologies.

In another aspect, the invention relates to the use of the method according to the invention for diagnosing a disease or for determining the susceptibility of a subject to a disease.

The invention also relates to a method for diagnosing a disease or for determining the susceptibility of a subject to a disease which comprises isolating nucleic acids according to the method of the invention.

“Diagnosing” as used herein, refers both to the process of attempting to determine and/or identify a possible disease in a subject, i.e. the diagnostic procedure, and to the opinion reached by this process, i.e. the diagnostic opinion. As such, it can also be regarded as an attempt at classification of an individual's condition into separate and distinct categories that allow medical decisions about treatment and prognosis to be made. As those skilled in the art will understand, such an evaluation, may not be correct for 100% of the subjects to be diagnosed, even though it preferably is correct for 100% of them. The term, however, requires being able to identify a statistically significant part of subjects as suffering from the disease. One skilled in the art can readily determine if a part is statistically significant using several well-known statistical evaluation tools, for example, determination of confidence intervals, determination of the p-value, Student's t-test, Mann-Whitney test, etc. or ROC analysis and the parameters with highest clinical utility the sensibility and specificity for classifying the person in the correct group.

Preferred confidence intervals are at least 50%, at least 60%, at least 70%, at least 80%, at least 90%, at least 95%. The p-values are, preferably, 0.05, 0.01, 0.005 or lower.

“Determining the susceptibility” or “determining the risk of suffering a disease”, as used herein, relates to a method for determining the probability that a patient suffers a disease.

The term “subject”, as used herein, refers to any animal classified as a mammal and includes, but is not limited to, pets or farm animals, primates and humans, for example, human beings, non-human primates, cows, horses, pigs, sheep, goats, dogs, cats, or rodents. Preferably, the subject is a human.

The invention also relates to the use of the method according to the invention for determining the prognosis or for monitoring the progression of a disease in a subject. The invention also relates to a method for determining the prognosis or for monitoring the progression of a disease in a subject which comprises isolating nucleic acids according to the method of the invention.

“Determining the prognosis”, as used herein, relates to the likelihood that a patient will have a particular clinical outcome, whether positive or negative. “Monitoring the progression of a disease”, as used herein, relates to the determination of one or several parameters indicating the progression of the disease in a patient suffering from a disease.

The invention also relates to the use of the method according to the invention for monitoring the effect of a therapy for the treatment of a disease. The invention also relates to a method for monitoring the effect of a therapy for the treatment of a disease in a subject which comprises isolating nucleic acids according to the method of the invention.

“Monitoring the effect of a therapy” relates to the response of the patient suffering from a disease to the therapy for treating said disease. Standard criteria (Miller, et al., Cancer, 1981; 47(1): 207-14) can be used herewith to evaluate the response to therapy including response, stabilization and progression, for example in cancer. The term “response”, as used herein, can be a complete response (or complete remission) which is the disappearance of all detectable malignant disease or a partial response which is defined as approximately >50% decrease in the sum of products of the largest perpendicular diameters of one or more lesions (e.g. tumor lesions), no new lesions and no progression of any lesion. Subjects achieving complete or partial response were considered “responders”, and all other subjects were considered “non-responders”. The term “stabilization”, as used herein, is defined as a <50% decrease or a <25% increase in tumor size. The term “progression”, as used herein, is defined as an increase in the size of tumor lesions by >25% or appearance of new lesions.

The term “therapy”, as used herein, refers to a therapeutic treatment, as well as a prophylactic or prevention method, wherein the goal is to prevent or reduce an unwanted physiological change or disease, such as cancer. Beneficial or desired clinical results include, but not limiting, release of symptoms, reduction of the length of the disease, stabilized pathological state (specifically not deteriorated), retard in the disease's progression, improve of the pathological state and remission (both partial and total), both detectable and not detectable.

The term “unfavourable clinical response”, as used herein, refers to not obtaining beneficial or desired clinical results which can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “favourable clinical response”, as used herein, refers to obtaining beneficial or desired clinical results which can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. The invention also relates to the use of the method according to the invention for identifying compounds suitable for the treatment of a disease. The invention also relates to a method for identifying compounds suitable for the treatment of a disease which comprises isolating nucleic acids according to the method of the invention.

“Compounds suitable for the treatment” refers to a screening method both for the identification of effective compounds for the treatment of the existing disease and for the preventive treatment (i.e., prophylaxis). The term “treatment” has been defined in the context of the methods for monitoring a therapy.

The invention also relates to the use of the method according to the invention for designing a personalized therapy in a subject or for selecting a patient susceptible to being treated with a therapy for the prevention and/or treatment of a disease. The invention also relates to a method for designing a personalized therapy in a subject or for selecting a patient susceptible to being treated with a therapy for the prevention and/or treatment of a disease in a subject in need thereof which comprises isolating nucleic acids according to the method of the invention.

As it is used herein, the expression “designing a personalized therapy” refers to the design and application of interventions for prevention and treatment adapted to the genetic substrate of the patient and for the molecular profile of the disease.

“Susceptible to being treated with a therapy”$means that there is a greater likelihood that the drug will be therapeutically efficacious against the disease compared to the likelihood of efficiency for a disease determined to be not “susceptible” to the agent. Determining that a disease is susceptible to treatment with a drug or drug class therefore provides a method for identifying a therapeutic regimen to treat the patient.

As previously described, experts in the field will understand that the methods of the invention, may not be correct for 100% of the subjects, even though it preferably may be correct for 100% of them.

As the person skilled in the art will understand the methods of the invention (e.g. for diagnosing a disease or for determining the susceptibility of a subject to a disease, for determining the prognosis or for monitoring the progression of a disease, for monitoring the effect of a therapy for the treatment of a disease in a subject, for identifying compounds suitable for the treatment of a disease, for designing a personalized therapy in a subject, or for selecting a patient susceptible to being treated with a therapy for the prevention and/or treatment of a disease in a subject in need thereof) comprise
a) isolating nucleic acids associated to or contained inside EVs from a sample of the subject according to the method of the invention,
b) analyzing the nucleic acids to determine their levels and characteristics, and
c) comparing the value of the data obtained in b) with a reference value.

The analysis of nucleic acids present in the EVs of step b) may be quantitative and/or qualitative. For quantitative analysis, the amounts (expression levels), either relative or absolute, of specific nucleic acids of interest within the EVs are measured with methods known in the art. For qualitative analysis, the species of specific nucleic acids of interest within or associated to the EVs, whether wild type or variants, are identified with methods known in the art.

Qualitative or quantitative alterations of nucleic acids associated to a disease include, without limitation, over-expression of a gene (e.g., oncogenes) or a panel of genes, under-expression of a gene (e.g., tumor suppressor genes such as p53 or RB) or a panel of genes, alternative production of splice variants of a gene or a panel of genes, gene copy number variants (CNV) (e.g. DNA double minutes), nucleic acid modifications (e.g., methylation, acetylation and phosphorylations), single nucleotide polymorphisms (SNPs), microsatellite instability, chromosomal and genes rearrangements (e.g., inversions, deletions, insertions, fusions and duplications), and mutations (insertions, deletions, duplications, missense, nonsense, synonymous or any other nucleotide changes) of a gene or a panel of genes, which mutations, in many cases, ultimately affect the activity and function of the gene products, lead to alternative transcriptional splicing variants and/or changes of gene expression level.

“Genomic alteration” or mutation, as used herein, relates to any alteration of the nucleotide sequence including substitutions, insertions, or deletions of small or large fragments of DNA, genomic amplification, and rearrangements. The determination of such genetic alterations can be performed by a variety of techniques known to the skilled practitioner. In general, the methods for analyzing genetic alterations are reported in numerous publications, not limited to those cited herein, and are available to skilled practitioners. The appropriate method of analysis will depend upon the specific goals of the analysis, the condition/history of the patient, and the specific cancer(s), diseases or other medical conditions to be detected, monitored or treated.

The skilled person in the art knows the particular nucleic acids to be analyzed in relation to a particular disease. For example in this context, specific exosomal miRNA signatures have been described, such as the miR-1246, miR-4644, miR-3976, and miR-4306 that were found upregulated in patients with pancreatic cancer or the overexpression of miR-211 in patients with BRAFV600 melanoma that correlated with reduced sensitivity to BRAF inhibitors. In addition, there are several EV-associated miRNA dysregulation detected in human diseases, such as miRNA-21 increased in hepatocellular carcinoma; miRNA-192, miRNA-30a, miRNA-122 increased in alcoholic hepatitis; miRNA-19b increased in prostate cancer patients; a multibiomarker panel (RNU6-1/miRNA-16-5p, miRNA-25-3p/miRNA-320a,let-7e-5p/miRNA-15b-5p,miRNA-30a-5p/miRNA-324-5p, miRNA-17-5p/miRNA-194-5p) increased in locally advanced esophageal adeno-carcinoma; miRNA-126, miRNA-199a increased levels inversely predict cardiovascular events; miRNA-375, miRNA-141p increased in prostate cancer; let-7a, miRNA-1229, miRNA-1246, miRNa-150, miR-21,miRNA-223,miRNA-23a increased in colon cancer; let-7f, miRNA-20b, miRNA-30e-3p decreased in non-small cell lung cancer; miRNA-1290, miRNA-375 higher levels associated with poor survival of prostate cancer; miRNA-1246 higher levels associated with aggressive form of prostate cancer; or miRNA-29c negatively associated with early renal fibrosis in lupus nephritis. Overexpression of mir-214, mir-140, mir-147, mir-135b, mir-205, mir-150, mir-149, mir-370, mir-206, mir-197, mir-634, mir-485-5p, mir-612, mir-608, mir-202, mir-373, mir-324-3p, mir-103, mir-593, mir-574, mir-483, mir-527, mir-603, mir-649, mir-18a, mir-595, mir-193b, mir-642, mir-557, mir-801, slet-7e, mir-21, mir-141, mir-200 are associated to ovarian cancer. Overexpression of mir-21, mir-146a relates to cervical cancer.

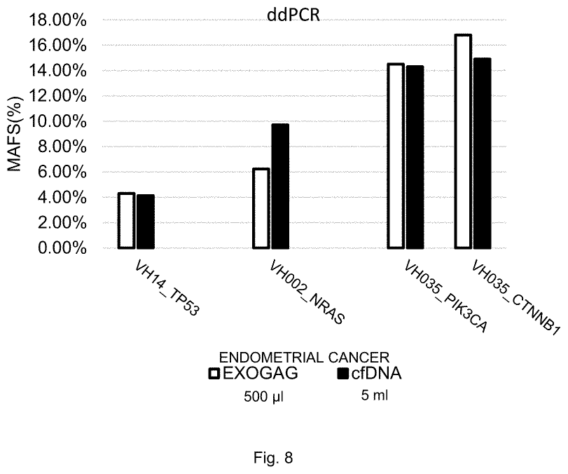

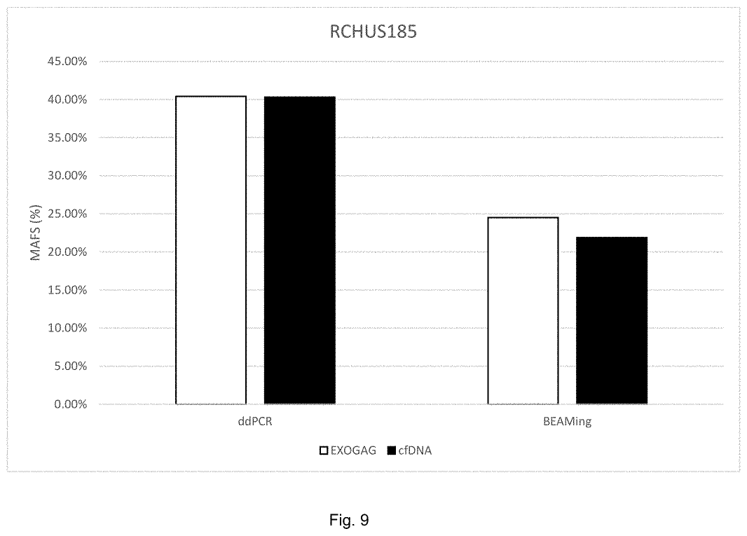

Alternatively it is possible to analyze the presence of a mutation or polymorphism. In this sense, for example, the identification of gene mutations in TP53, NRAS, PIK3CA and CTNNB1 genes has already been proved in patients with endometrial cancer in DNA obtained from EVs (FIG. 8) and KRAS point mutations in patients with colorectal cancer (FIG. 9). In relation to the response to a treatment, there are also known several sequences that can be analysed related to a particular response. For example, a high expression of programmed cell death 1 and CD28 molecules by T-cell derived-EXO (TEX) at baseline predicts the response to ipilimumab, a cytotoxic T-lymphocyte antigen 4 (CTLA4) inhibitor, in patients with metastatic melanoma. Similarly, CD80 and CD86 levels on dendritic cell derived EXO (DEX) reflect the restoration of antimelanoma activity from the immune system, thus supporting both TEX and DEX as reliable prognostic biomarkers in melanoma.

Once analyzed the nucleic acids to determine a genomic alteration or quantification, the methods further comprise comparing the value of the data obtained in b) with a reference value.

As it is used herein, the term “reference value” refers to a value obtained in the laboratory and used as a reference for the values or data obtained by means of laboratory examinations of the patients or samples collected from the patients. The reference value or reference level can be an absolute value, a relative value, a value having an upper and/or lower limit, a range of values, a mean value, a median value, or a value compared to a specific control or reference value. The reference value can be based on a value of the individual sample, such as a value obtained from a sample of the subject being tested, for example, but at an earlier time. The reference value can be based on a large number of samples, such as the values of the population of subjects from the same age group, or can be based on a set of samples, including or excluding the sample to be tested.

“Disease”, as used herein relate to an abnormal condition affecting the body of an organism. The term also refers to any type of disease that can be diagnosed by analyzing nucleic acids, such as those of genetic origin, including rare diseases. The term also refers to a disorder which relates to a functional abnormality or disturbance. Illustrative, non-limitative examples of diseases are cancer, cutaneous conditions, endocrine diseases, eye diseases, intestinal diseases, infectious diseases, liver diseases or heart diseases.

In a preferred embodiment of the uses and methods of the invention, the disease is cancer.

The term “cancer”, as used herein, refers to a disease characterized by uncontrolled cell division (or by an increase of survival or apoptosis resistance) and by the ability of said cells to invade other neighbouring tissues (invasion) and spread to other areas of the body where the cells are not normally located (metastasis) through the lymphatic and blood vessels, circulate through the bloodstream, and then invade normal tissues elsewhere in the body. Depending on whether or not they can spread by invasion and metastasis, tumours are classified as being either benign or malignant: benign tumours are tumours that cannot spread by invasion or metastasis, i.e., they only grow locally; whereas malignant tumours are tumours that are capable of spreading by invasion and metastasis. As used herein, the term cancer includes, but is not limited to, the following types of cancer: breast cancer; biliary tract cancer; bladder cancer; brain cancer including glioblastomas, in particular glioblastoma multiforme, and medulloblastomas; cervical cancer; head and neck carcinoma; choriocarcinoma; colon cancer, colorectal cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia/lymphoma; intraepithelial neoplasms including Bowen's disease and Paget's disease; liver cancer, hepatoma; lung cancer, pleural mesothelioma; lymphomas including Hodgkin's disease and lymphocytic lymphomas; neuroblastomas; oral cancer including squamous cell carcinoma; parotid gland cancer; ovarian cancer including those arising from epithelial cells, stromal cells, germ cells and mesenchymal cells; pancreatic cancer; prostate cancer; kidney cancer, suprarenal cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; cervix cancer, endometrial cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Other cancers will-be known to one of ordinary skill.

In a more preferred embodiment, the cancer is endometrial cancer or colorectal cancer.

The methods of the invention are carried out “in vitro”, i.e., they are not carried out to practice on a human or animal body.

All the terms and embodiments previously described are equally applicable to this disclosure.

Kits and Uses Thereof

In another aspect, the invention relates to a kit comprising dimethylmethylene blue (DMB) and a reagent capable of isolating nucleic acids from EVs.

As it is used herein, the term “kit” refers to a combination of a set of reagents suitable for separating and/or isolating nucleic acids associated to or contained inside EVs. The kit optionally includes other types of biochemical reagents, containers, packaging suitable for commercial sale, electronic hardware and software components, etc. The reagents are packaged to allow for their transport and storage. Materials suitable for packaging the components of the kit include glass, plastic (polyethylene, polypropylene, polycarbonate, and the like), bottles, vials, paper, sachets, and the like. Additionally, the kits of the invention may contain instructions for the simultaneous, sequential, or separate use of the different components in the kit. Said instructions can be found in the form of printed material or in the form of an electronic support capable of storing instructions such that they can be read by a subject, such as electronic storage media (magnetic disks, tapes, and the like), optical media (CD-ROM, DVD), and the like. Additionally or alternatively, the media may contain internet addresses which provide said instructions.

In a preferred embodiment, the kit comprises dimethylmethylene blue (DMB) at a concentration comprised between 0.01 and 100 nM at a pH comprised between 2 and 6.9.

In a preferred embodiment, the pH is comprised between 3 and 4; more preferably comprised between 3.5 and 4; more preferably between 3.3 and 3.6; more preferably it is 4. In a preferred embodiment, the pH measured at 20° C. is 3.5.

In another preferred embodiment, the concentration of DMB is comprised between 0.29 and 0.35 mM, more preferably 0.29 mM or 0.30 mM, even more preferably 0.29 mM, and wherein the pH is comprised between 3.3 and 3.6, preferably comprised between 3.5 and 4.

“Reagent capable of isolating nucleic acids from EVs”, as used herein, relates to any reagent to isolate nucleic acids associated to or inside the EVs. In a preferred embodiment, the reagent is capable of isolating DNA, such as phenol, chloroform or commercial reagents. In another preferred embodiment, the reagent is capable of isolating RNA. The reagent may be organic or inorganic. In a preferred embodiment, the reagent is Trizol or RIPA.

The kit of the invention may also include a variety of buffers including loading and wash buffers. Loading and wash buffers can be of high or low ionic strength. The buffers may include one or more of the following components: Tris, Bis-Tris, Bis-Tris-Propane, Imidazole, Citrate, Methyl Malonic Acid, Acetic Acid, Ethanolamine, Diethanolamine, Triethanolamine (TEA) and Sodium phosphate. Detergents include, but are not limited to, sodium dodecyl sulfate (SDS), Tween-20, Tween-80, Triton X-100, Nonidet P-40 (NP-40), Brij-35, Brij-58, octyl glucoside, octyl thioglucoside, CHAPS or CHAPSO.

In another aspect, the invention relates to the use of a kit comprising DMB or the kit according to the invention for isolating nucleic acids associated to or contained inside EVs.

All the terms and embodiments previously described are equally applicable to this disclosure.

The invention will be described by way of the following examples which are to be considered as merely illustrative and not limitative of the scope of the invention.

Materials and Methods

Isolation of EVs

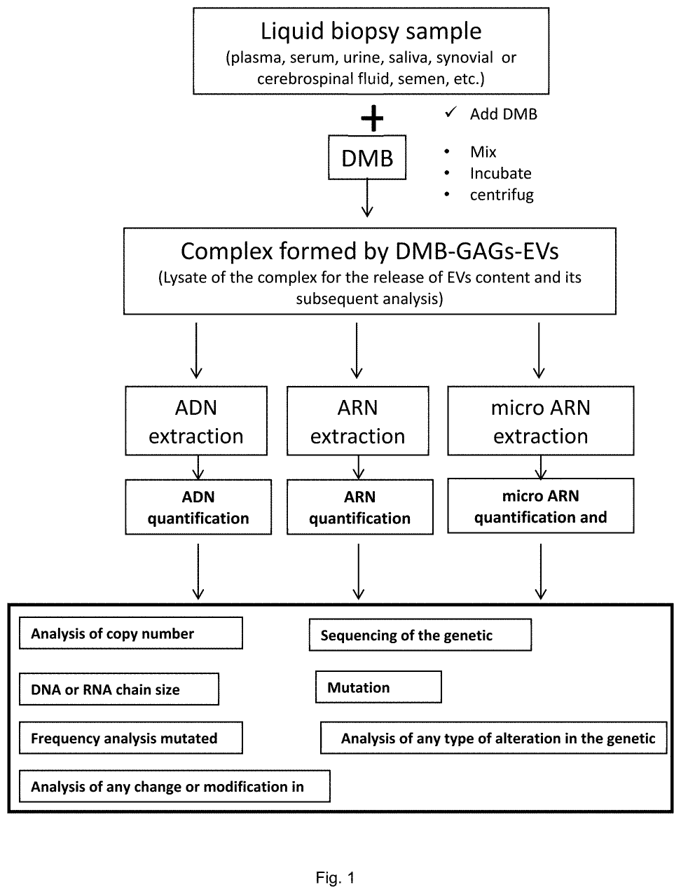

FIG. 1 shows the protocol for isolating nucleic acids using DMB, 1,9-Dimethylmethylene Blue zinc chloride double salt, after a brief incubation, to bind to the complex formed by GAGs-EVs.

The isolated DNA, RNA or microRNA may be subjected to quantitative and qualitative analyses including the determination of the size of both DNA and RNA chains, the levels of specific DNA or RNA sequences (gene expression), the number of gene copies, the analysis of different classes of mutations including any alteration of the nucleotide sequence such as substitutions, insertions, or deletions of small or large fragments of DNA, genomic amplification, and rearrangements or any technique that allows analyzing any change or modification that occurs in the genetic material (at genomic, transcriptomic and epigenomic level). These analyses can be applied to the field of biomedicine as this information can be used to predict, detect, or monitor different pathologies.

For the EVs isolation, DMB, 1,9-Dimethylmethylene Blue zinc chloride double salt (Sigma-Aldrich) molecule was used at a concentration of 0.30 mM, in a solution composed by glycine and sodium chloride dissolved in acetic acid at a 0.1 M concentration, into a final solution of 0.01 M acetic acid concentration. DMB precipitation solution has a final pH between 3.5 and 4, since in this context the solution has a positive charge with the ability to bind to EVs by having a negative charge (due to the negative charge of GAGs containing EVs).

In this way the DMB-EVs complexes are unstable in solution and precipitate and in this way and in a very simple way it is possible to isolate the EVs from a liquid biopsy sample when the DMB-GAGs-Exosome junction or complex is made.

Human Samples Collection

Conditioned Medium Collection (Secretome or Cell Culture Medium)
Hec1A cell line was cultured in McCoy's 5A media (Gibco, Grand Island, N.Y., USA) supplemented with 10% FBS (Gibco, South America) depleted of EV and 1% penicillin-streptomycin (Gibco, Grand Island, N.Y., USA), at 37° C. and 5% CO₂. After 48 h, the culture medium was recovered for exosome isolation.

Human Plasma Collection
Patients with endometrial cancer participating in the study were recruited at the Gynecologic Department of Vail d'Hebron University Hospital (Barcelona, Spain), the MDA Anderson Cancer Centre of Madrid and the University Hospital of Santiago de Compostela (Santiago de Compostela, Spain) under fully informed consent and ethical approval by the Galician Ethical Committee (reference: Code 2017/430). Patients with CRC participating in the study were recruited at the University Hospital of Santiago de Compostela (Santiago de Compostela, Spain) under fully informed consent and ethical approval by the Galician Ethical Committee (reference: Code 2015/744).
Peripheral blood from patients was collected in CellSave tubes (Menarini, Silicon Biosystem, Huntingdon Valley, USA) or Streck (Streck, La Vista, Nebr.). Plasma was then extracted after two steps of centrifugation at 1600 g and 6000 g during 10 min. After the second centrifugation plasma was stored at −80° C. until use.

Human Urine Collection
Urine from healthy donors was collected in sterile conditions. Urine samples were sequentially centrifuged (300 g, 10 minutes; 800 g, 15 minutes; 10.000 g, 30 minutes) and filtered (0.22 μm) before used or frozen.

Ascitic Fluid Collection
Ascites fluid from advanced stage III/IV ovarian cancer patients (n=9) was collected in sterile conditions at the Medical Oncology Department at the University Hospital of Santiago de Compostela (Spain) under fully informed consent and ethical approval by the Galician Ethical Committee (reference: 2014/309). Ascites samples were sequentially centrifuged (300 g, 10 minutes; 800 g, 15 minutes; 10.000 g, 30 minutes) and filtered (0.22 μm).

Saliva Collection
Saliva samples were collected and processed as described previously (Majem B, Li F, Sun J, Wong D T. RNA Sequencing Analysis of Salivary Extracellular RNA. Methods Mol Biol. 2017; 1537:17-36.) Unstimulated whole saliva samples were collected from the participants between 9 and 10 am, before any therapeutic procedures. Subjects were refrained from eating, drinking and oral hygiene procedures for at least 1 hour before the collection. Subjects rinsed their mouth with distilled water to minimize contamination of the salivary samples. Five minutes after the oral rinsing, the participants started spit into a 50-mL Falcon tube kept on ice. As minimum, five milliliters of saliva were collected from each participant. Immediately after collection, salivary samples were centrifuged at 2600 g for 15 minutes at 4° C. to remove cellular components. Saliva supernatant was then separated from pellet and 1 μL per mL of supernatant saliva of RNase inhibitor (SUPERase-In, AM2694, Ambion, Life Technologies) was added. All samples were aliquoted in 1,200 μL and stored at −80° C. prior to assay.

Exosome Isolation Method (Example Protocol)
1. Collect the plasma/serum/urine sample. Samples can be frozen until the moment in which they are used; if the sample has been frozen, thaw it and temper it before processing.
2. Centrifuge the sample at 2000×g for 10 minutes to remove cells and cell debris.
3. Transfer the supernatant to a new tube and discard the pellet of possible cell debris.
4. Add the volume of sample to isolate EVs to a new tube and add twice the volume of reagent A. That is, use a sample/reagent A ratio 1:2; (for example, to process 500 μl of plasma, add 1000 μl of precipitation reagent).
5. Mix the sample and precipitation reagent by inverting the tube or vortexing to homogenize the final solution (the solution will have a characteristic blue color).
6. Incubate the sample for 5 minutes at 4° C.
7. Centrifuge the sample at 16.000×g; 15 minutes at 4° C.
8. Remove the supernatant being careful not to remove the pellet containing the EVs (this pellet will be dark blue).
9. Resuspend the EVs in the appropriate buffer, depending on the technique and analysis, as protein analysis (mass spectrometry analysis, western-blot, Elisa, protein arrays, etc.) or genetic material analysis (ADN, ARN or micro RNA based analysis as PCR, digital PCR, BEAMing, sequencing, etc.).

Nano tracking analytical particle (NTA) technology of isolated EVs.

EVs from 50 μl of plasma were collected by DMB as described in this invention. Once the EVs were isolated, they were resuspended in a total volume of 1 ml of particle-free PBS so that there would be not interference with the quantification by NTA. The sample was passed by the NTA nanosight NS300 (Malvern, UK), which consists of a cytometer that is able to measure the Brownian movement of particles that move in a fluid.

Western Blot

50 μl of plasma (1), urine (2) or ascites liquid (3) was used for the EVs isolation as described in this invention and following the EVs isolation methodology. After isolation, EVs were lysed with a RIPA protein lysis buffer containing protease inhibitors to release their content.

The EVs precipitate after its lysate was boiled for denaturation at 95° C. for 5 minutes in Laemli buffer with p-mercaptoethanol. 40 μl were loaded on a 7% SDS-PAGE polyacrylamide gel and the proteins were separated for 1 hour and 30 minutes at 80V. Subsequently, the gel was transferred by wet transfer to a PVDF membrane for 1 hour and 30 minutes.

Membrane was incubated with a biotinylated anti-CD9 primary antibody overnight at 4° C. and subsequently incubated with an anti-streptavidin-HRP secondary antibody to visualize the signal of the CD9 protein used as an exosome marker.

Electron Microscopy

EVs isolated from urine (a), and plasma (b, c, d) were visualized by electron microscopy, according to the protocol which is described in this invention.

Once isolated, EVs were suspended in 50 μl of an isotonic saline buffer (PBS). The sample was diluted 1:1000 to observe the dispersed EVs since their concentration is very high. From this sample only 20 microliters were collected and deposited on a carbon grid (carbon film, mesh copper; CF400-CU). The sample was incubated for 5 minutes and then the remaining sample was removed with a blotting paper.

One sample was dried for 1 hour and analyzed using electron microscope to visualize the EVs contained in the sample.

A second sample was incubated with the mouse anti-CD9 antibody at a 1:1000 dilution for 1 hour at room temperature. Subsequently, it was incubated for 1 hour with a secondary anti-mouse antibody labeled with gold particles (to be able to visualize it by contrast in the electron microscope), and finally the sample was dried for 1 hour and visualized in the electron microscope to see if the EVs express the CD9 exosome marker.

DNA Extraction and Quantification from EVs Isolated by DMB

To evaluate the amount of genetic material that is extracted by the described method based on DMB in this invention, EVs were isolated from 500 μl of plasma from 6 endometrial cancer patients.

After isolation, DNA extraction from EVs was performed by DNeasy blood and tissue kit (Qiagen) which contains a potent lysis buffer able to lysate EVs and release their genetic content, and subsequently, the DNA that was associated or inside the EVs was quantified by fluorometry (Qubit).

On the other hand, DNA extraction from 5 ml of plasma was performed using the

QIAamp DNA Circulating Nucleic Acid Kit (Qiagen, Venlo, Netherlands) according to the manufacturers instructions, which contains a dissociation buffer (no lysis buffer). Therefore, only cell free-circulating DNA, but not the DNA which is inside the EVs, was isolated, because EVs are not lysed using this buffer. In this way the inventors extracted DNA from the EVs fraction (after DMB reaction) isolated from 500 μl of plasma and compared it with the amount of cell free DNA obtained from 5 mL of plasma.

### Example 1—Graphic Description of the Method of EVs Isolation by Using DMB and the Possibilities of Analysis by Different Approaches that are Compatible with the Technique and that Allow the Analysis of the Genetic Material Contained and Associated to the Isolated EVs

To assess the efficiency of EVs isolation, different samples were tested, as plasma and cell culture medium (secretomes).

Different isolation conditions were tested to evaluate efficiency of the DMB-GAGs-EVs complex isolation (Table 1).

Culture media was evaluated on the one hand, as the technique being refined, since these are samples of low complexity and very easily evaluable.

Cell culture media depleted of EVs was collected after 48 hours. Plasma samples were collected and processed as previously described.

The inventors started evaluating different centrifugation times, different precipitation forces (this is very important because centrifuges that reach 13000 g only allow handling very small sample volumes (maximum 2 mL), which does not make it possible to isolate many types of samples such as culture media; while those that reach 3500 g allow to process a large volume of sample). On the one hand the inventors could check lower the precipitation time necessary to isolate the EVs up to 5 minutes without compromising efficiency and on the other hand the inventors could decrease the precipitation rate as long as they increase the time up to 30 minutes.

Besides, different centrifugal temperature conditions were evaluated (since many of the centrifuges used in laboratories and/or hospitals do not have this option), and the inventors did not observe a significant difference, so it seemed reasonable to establish as a modification of the protocol the centrifugation at 4° C. Table 1 shows the results of the different isolation conditions tested.

TABLE 1
Conditions to obtain appropriate conditions for the maximum efficiency
in EVs isolation. (EVs from culture medium were enriched by
ultracentrifugation previously due to the high volumen).

| | Ratio | | | | | | Efficiency |
|---|---|---|---|---|---|---|---|
| | Sample/ | Incubation | Incubation | Precipitation | Precipitation | Precipitation | (particles/ |
| Sample | reactive | time | T^{a} | Time | Force | T^{a} | frame) |
| plasma | 1:0.1 | 5′ | 4° C. | 15′ | 16000 × g | 4° C. | 6.8 |
| plasma | 1:0.5 | 5′ | 4° C. | 15′ | 16000 × g | 4° C. | 14.6 |
| plasma | 1:2 | 5′ | 4° C. | 15′ | 16000 × g | 4° C. | 105.4 |
| plasma | 1:10 | 5′ | 4° C. | 15′ | 16000 × g | 4° C. | 44.5 |
| Culture | 1:0.5 | 5′ | 4° C. | 15′ | 16000 × g | 4° C. | 60.3 |
| media | | | | | | | |
| Culture | 1:2 | 5′ | 4° C. | 15′ | 16000 × g | 4° C. | 30.8 |
| media | | | | | | | |
| Culture | 1:0.5 | 5′ | 4° C. | 15′ | 16000 × g | 4° C. | 116.3 |
| media | | | | | | | |
| Culture | 1:0.5 | 5′ | 4° C. | 30′ | 4000 × g | 4° C. | 89.9 |
| media | | | | | | | |
| Culture | 1:0.5 | 5′ | 4° C. | 2′ | 16000 × g | 4° C. | 90.8 |
| media | | | | | | | |
| Culture | 1:0.5 | 5′ | 4° C. | 2′ | 16000 × g | 4° C. | 91.1 |
| media | | | | | | | |
| Culture | 1:0.5 | 15′ | 4° C. | 2′ | 16000 × g | 4° C. | 69.2 |
| media | | | | | | | |
| Culture | 1:0.5 | 1′ | TA | 2′ | 16000 × g | 4° C. | 69.7 |
| media | | | | | | | |
| Culture | 1:0.5 | 2′ | 4° C. | 2′ | 16000 × g | 4° C. | 84.3 |
| media | | | | | | | |
| Culture | 1:0.5 | 2′ | TA | 2′ | 16000 × g | TA | 79.2 |
| media | | | | | | | |
| Culture | 1:0.5 | 5′ | 4° C. | 5′ | 16000 × g | 4° C. | 79.6 |
| media | | | | | | | |
| Culture | 1:0.5 | 5′ | TA | 5′ | 16000 × g | TA | 97.3 |
| media | | | | | | | |

Once the time and temperature conditions in the precipitation process of the EVs were evaluated, the inventors tested them in the process of incubation of the reagent with the sample.

### Example 2—NTA Nanosight NS300 Particle Tracking Profile of Plasma EVs Isolated by DMB and Ultracentrifugation

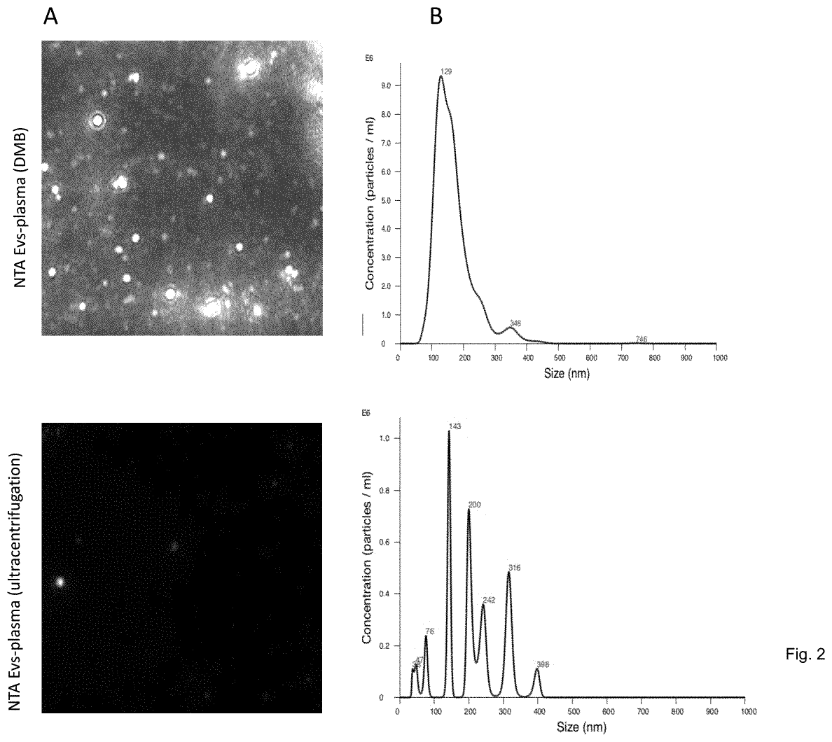

FIG. 2A shows a representative image of video recorder from EVs particles from plasma isolated by DMB (upper panel) and ultracentrifugation (lower panel). FIG. 2B shows a representative image of EVs isolated by DMB (upper panel) and ultracentrifugation (lower panel), expressed as particles size (nm) and concentration (particles/ml).

### Example 3—Exosome Characterization by Western-Blot

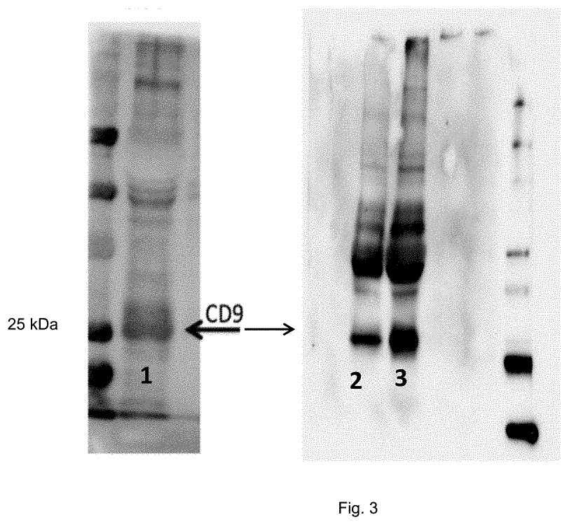

FIG. 3 shows the presence of one of the most commonly used markers for the characterization of EVs, CD9, analyzed by western blot, in plasma sample (1), urine (2) and ascites fluid (3). The levels of the marker can be seen due to the band at the height of 25 kDa (black arrow).

### Example 4—Visualization of Isolated EVs by Transmission Electron Microscopy (TEM)

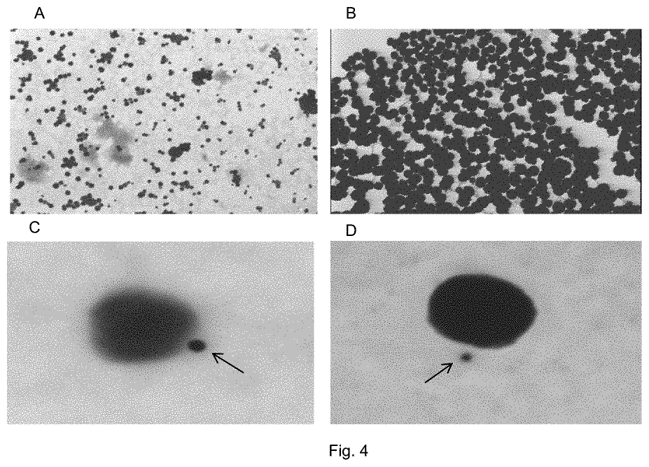

FIG. 4A shows transmission electron microscopy (TEM) of EVs isolated by DMB technique from urine. FIG. 4 B shows transmission electron microscopy (TEM) of EVs isolated by DMB technique from plasma. FIGS. 4 C and D show EVs isolated from plasma incubated with the anti-CD9 antibody. Exosomes express the CD9 exosome marker after immunogold staining. The signal is located in the exosome membrane since this marker is a membrane marker. On the other hand, the size of the obtained exosomes is over 100-150 nm that are within international standards classifying extracellular vesicles as exosomes.

### Example 5—Efficiency and Purity of EVs from Culture Media Isolated by DMB and Ultracentrifugation

EVs obtained from culture media were isolated by DMB, as previously described, and ultracentrifugation.

Samples were passed through NTA nanosight NS300 (Malvern, UK), and the efficiency of EVs isolated by DMB, expressed as number of EVs/frame, was similar than that obtained by ultracentrifugation (FIG. 5A). By contrast, quantification of protein contaminants showed low co-precipitated protein levels in EVs isolated by DMB compared to those isolated by ultracentrifugation (FIG. 5B).

### Example 6—Purity Analysis of Isolated EVs

An assay has been established for the evaluation of the purity of EVs, that is, a method based on the quantification of co-precipitated material during the isolation process, but not associated with EVs.

EVs were isolated from 50 μl of human plasma with the technology described in this invention (DMB), and by two commercial technologies as ExoQuick® (System Biosciences, Mountain view, CA) and Exo-Spin™ (Cell Guidance Systems, Cambridge, UK). ExoQuick® is based on a polymer (PEG) that precipitates the EVs; and Exo-Spin™ combines precipitation with a polymer and size exclusion chromatography.

Isolation made by ExoQuick® (ExoQuick) (Chugh P E et al., PLoS Pathog. 2013. 9(7): e1003484) and Exo-Spin™ (Li Z. et al., Molecular and Cellular Biochemistry 439(1-2):1-9.) (ExoSpin) was carried out following the manufacturers instructions starting from the same volume of plasma as in the other conditions (50 μl). Total protein measurement was made by a detection of co-precipitated protein, that is, protein that each technique has precipitated but that are within the EVs, that's mean they are precipitated contaminating protein due to the low specificity of each methodology.

Co-precipitated proteins were measured using Bradford assay (as manufacturer) in a native pellet, without lysis buffer, for avoiding the releasing of EVs cargo. Therefore, the inventors only measured free proteins or those binding the EVs membrane.

FIG. 6 shows that the co-precipitation of proteins and EVs using DMB is minimal, compared to the other methodologies (ExoQuick and ExoSpin).

These results indicated that EVs isolation technology of the invention is cleaner than commercial competitors as ExoQuick® and Exo-Spin™.

### Example 7—Extraction and Quantification of DNA from EVs Isolated by DMB and Cell-Free DNA from Plasma

The upper graph of FIG. 7 (7A) represents the levels of DNA obtained in 6 endometrial cancer patients (VH14, VH002, VH035, BL24, BL15 and 964) from EVs isolated using DMB from plasma and from non-processed plasma samples. The Y axis represents the DNA concentration (ng/μL) of each of the patients. For obtaining the DNA associated to the EVs-DMB, 500 μL of plasma were used (white column) while for cell-free DNA (cfDNA) (black column) 5 mL of plasma were used (10 times more volume of plasma). DNA concentration was measures by Qubit technology. cfDNA was obtained by the QIAamp Circulating Nucleic Acid Kit (QIAGEN).

This comparison was addressed to determine the advantage of using EVs-DNA obtained with DMB (EXOGAG) instead of cfDNA in terms of reduction of the volume of plasma required to attempt different genetic analyses. Of note, the amount of plasma is always limited and critical for genetic studies.

As can be seen in FIG. 7 below (7B), the average amount of DNA extracted using both methodologies is similar, but using EVs-DNA after DMB precipitation considerable lower volume is required (10 times less volume of plasma) than that required for cfDNA analyses.

### Example 8—Characterization of DNA-EVs Isolated by DMB

The size distribution of DNA-EVs isolated by DMB using plasma (500 μL) (Table 3) and cfDNA obtained from 5 mL of total plasma (Table 2) was analyzed using Agilent 2200 TapeStation. Normalization was performed using two internal markers, visible as extreme amplitudes at 25 and 1,500 bp, respectively. Plasma sample was obtained from a patient with colorectal cancer. Importantly, in both cases the average size of the DNA obtained was around 150 bp as was expected for cfDNA and EVs-DNA.

TABLE 2
Size distribution of cfDNA obtained from 5 mL of total plasma.

| Size | | Peak Molarity | % Integrated | |
|---|---|---|---|---|
| (bp) | Conc. [pg/μL] | [pmol/l] | Area | Observations |
| 25 | 978 | 60200 | — | Lower Marker |
| 155 | 12800 | 127000 | 100.00 | cfDNA |
| 1500 | (250) | 256 | — | Upper Marker |

TABLE 3
Size distribution of DNA-EVs isolated by DMB using 500 μl of plasma.

| Size | Cone. | | | |
|---|---|---|---|---|
| (bp) | [pg/μL] | Peak Molarity [pmol/l] | % Integrated Area | Observations |
| 25 | 518 | 31900 | — | Lower Marker |
| 149 | 2600 | 26700 | 61.11 | evDNA |
| 680 | 1650 | 3740 | 38.89 | evDNA |
| 1500 | (250) | 256 | — | Upper Marker |

### Example 9—Detection of Point Mutations is Feasible Using EVs-DNA Isolated with DMB

The analysis of point mutations with clinical value was addressed in both EVs-DNA and cfDNA in 3 endometrial cancer patients by ddPCR (Biorad QX200 technology). The percentage of the mutant allele fraction (MAFs) for each mutation is represented in FIG. 8.

Importantly, the MAFs levels detected were comparable using both EVs-DNA (white bars) or cfDNA (black bars), but using the method of the invention lower volumes of plasma were required. 10 times less volume of a patient's plasma was required for the analysis of point mutations with clear clinical relevance in EVs-DNA isolated with DMB, which is a very important milestone since the sample volume in cancer patients is critical. Therefore, the use of DMB technology allows a more rational use of the plasma samples and the possibility to perform additional analyses.

### Example 10—Detection of Point Mutations is Feasible by Digital PCR and BEAMing Application on EVs-DNA Isolated with DMB

The levels of KRAS point mutations were quantified in EVs-DNA isolated with DMB (method described in this invention) from 500 μL of plasma from one patient with colorectal cancer (RCHUS185) and using cfDNA obtained from 5 mL of total plasma.

After the DNA isolation, the analysis of point mutations was addressed by ddPCR and BEAMing (a technique used in many Hospitals to detect mutations in cfDNA) to evaluate and compare the performance of both techniques to characterize EVs-DNA isolated with DMB.

FIG. 9 represents the mutant allele fraction (MAFs) of the point mutation (in KRAS) analyzed by each technology using EVs-DNA (EXOGAG) and cfDNA. MAFs levels were comparable between the two analytical technologies and the DNA sources, evidencing that the invention method is compatible with BEAMing technology, used nowadays for the clinical routine to determine RAS/BRAF mutations. Besides, it is important to highlight that using the invention method 10 times less volume of a patient's plasma was required for the analysis of point mutations with clinical relevance.

### Example 11—Evaluation by Nano-Tracking Analytical Particle (NTA) Technology of Isolated EVs from Saliva

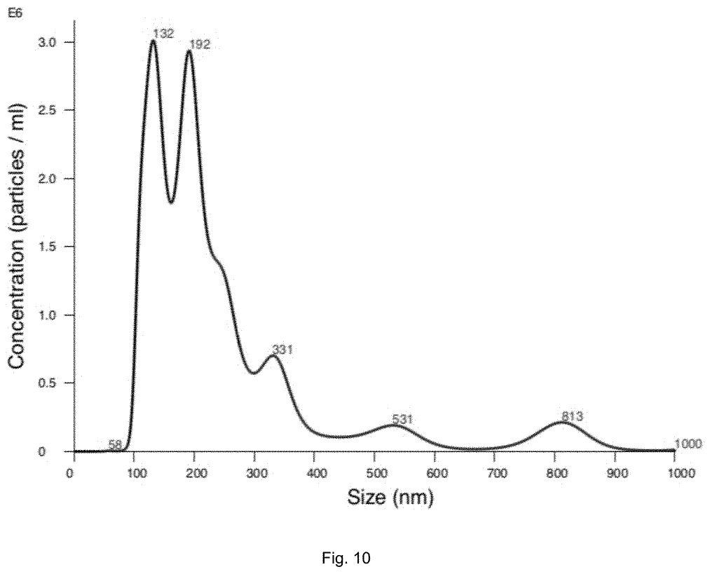

Frozen saliva samples were thawed thoroughly on ice and centrifuged 10,000×g at 4° C. for 5 minutes to eliminate cell debris saliva. Then, 500 μL-saliva were diluted 1:2 with DMB and centrifuged at 16,000×g for 15 min at 4° C. The resulting supernatant was removed, and pellet was resuspended in 500 μl of PBS particle free. For RNase A treatment, EVs were incubated with 500 μL of 0.1 mg/mL RNase A (Qiagen) for 1 hour at 37° C. Once the EVs were isolated, sample was resuspended in a total volume of 1 mL of particle-free PBS. The sample was analyzed by the NTA nanosight NS300 (Malvern, UK). FIG. 10 shows profile and size of EVs isolated from saliva using DMB. Profile and size were very similar, around 150 nm, which confirms that with the method of precipitation of EVs by DMB described in this invention, EVs fitting the standards set by the scientific community.

### Example 12—RNA and microRNA Quantification from Saliva EVs Samples Using the DMB-Based Precipitation Technique

Total RNA containing miRNAs (miRNeasy extraction Micro Kit, Qiagen, Hilden, Germany) was extracted from EVs samples isolated using DMB from saliva. EVs fraction was lysed in 750 μL of Trizol LS Reagent (10296-028, Ambion, Life Technologies). Thereafter, 200 μL chloroform was added to the denatured saliva and mixed by vortex for 30 seconds, followed by an incubation for 5 minutes at room temperature. The addition of chloroform causes phase separation where protein is extracted to the organic phase, DNA resolves at the interface, and RNA remains in the aqueous phase. Total RNA was eluted from the spin column membrane in 60 μL pre-heated RNA-free water (50° C.), and DNase treatment (DNase, Roche) was used to remove contaminating DNA during RNA extraction. After RNA precipitation, the final RNA was suspended in 10 μL pre-heated RNA-free water (50° C.), then incubated for 5 minutes at 55° C. and RNA samples were stored at −80° C. for further analyses. RNA concentration was measured by Quantus™ Fluorometer (Promega). RNA integrity was assessed by a Bionalyzer (Agilent, Santa Clara, Calif.) using the Small RNA Kit (Agilent). For normalization of sample-to-sample variation during RNA isolation and as internal control, same amounts (3.5 μL of 1.6×10⁸ copies/μL) of synthetic C. elegans miRNA-39 (cel-miR-39) was added into each denatured sample.

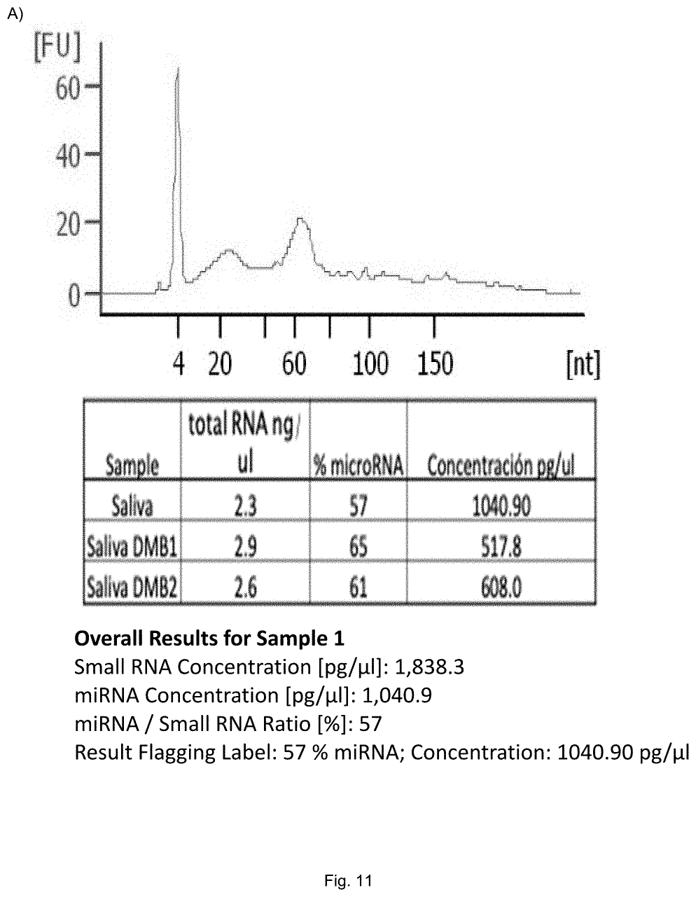

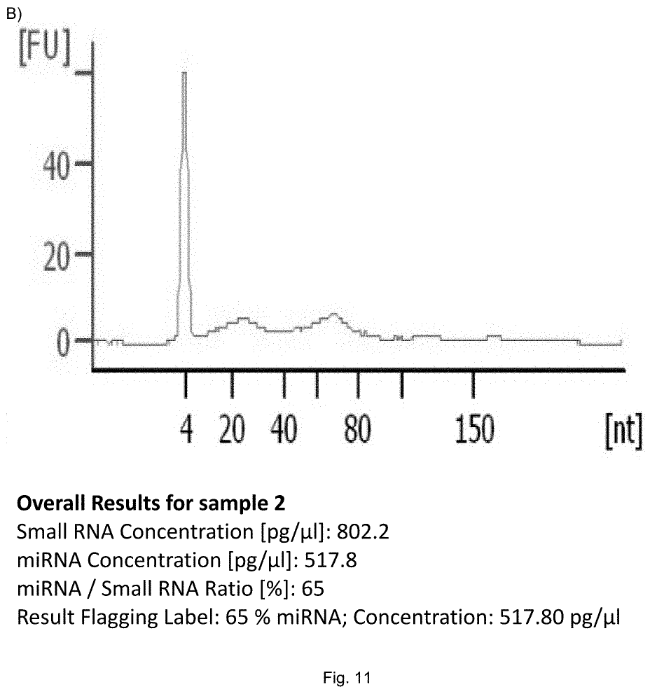

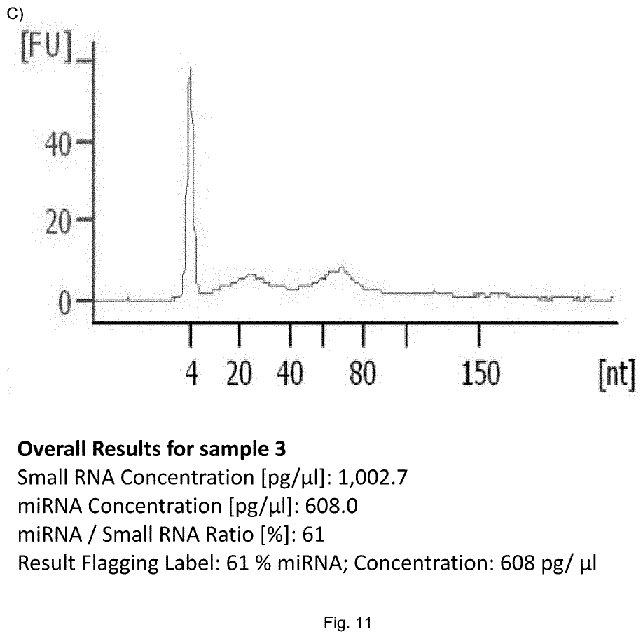

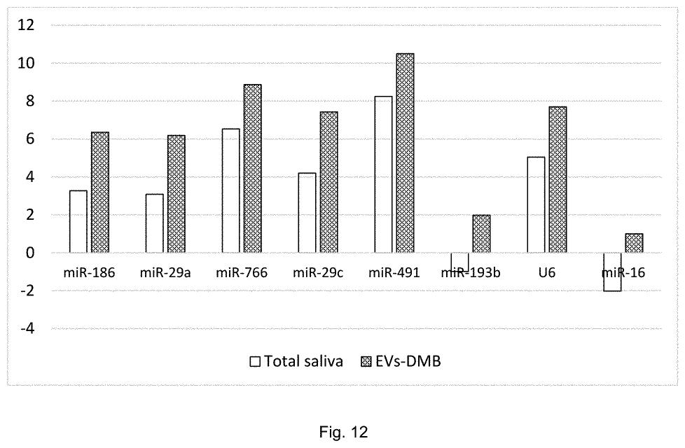

Total yield of RNA was comparable using the EVs fraction than total saliva. RNA levels using total saliva were 2.3 ng/μl, while after isolation of EVs with DMB whose performance ranges from 2.9 and 2.6 ng/μl (see FIG. 11).

Concentration of small RNA and micro RNA was lower in total saliva (FIG. 11 above left) than in EVs extracted from saliva using DMB (FIG. 11 below), in these samples.

However, despite the lower concentration of small and micro RNAs, the EVs fraction was enriched in microRNAs since they represent a higher percentage of the genetic material.

### Example 13—miRNA Expression Analysis by RT-qPCR Assay in EVs Isolated by DMB

FIG. 11 shows the profile of different microRNAs, using an exogenous microRNA (cel-miR-39) as a normalizer. The general profile of analyzed microRNAs was similar, but their levels were lower in total saliva compared with those in saliva EVs obtained using the DMB technique of the invention.

These results demonstrated that RT-qPCR technique is suitable for microRNA quantification in saliva samples using DMB for EVs isolation.

### Example 14—Analysis of DNA Associated with EVs and No-Co-Precipitation of Cell Free DNA (cfDNA)

Several conditions were tested to evaluate the amount of cfDNA, and to assess whether the genetic material contained in EVs using DMB is predominantly associated with exosome genetic material. The conditions are explained as follows.

Condition 1: The inventors started from 250 μL of plasma to which they added 250 μl of reaction buffer (RB) and 25 μl of DNase (baseline Zero DNase, LUCIGEN, cat No DM0715K), following the manufacturer instructions. After an incubation of 30 minutes at 37° C., the inventors proceed to isolate the EVs according to the DMB methodology described in this invention.

Condition 2: The inventors started from 250 μl of plasma to which they added 250 μl of reaction buffer (RB) and 25 μL of nuclease free water (NFW). After an incubation of 30 minutes at 37° C., the inventors proceeded to isolate the EVs according to the DMB methodology described in this invention.

Condition 3: The inventors started from 250 μl of plasma and 10,000 copies of the AKT p.E17K mutation (gBlock) to which they added 250 μl of reaction buffer (RB) and 25 μl of the DNase (baseline) Zero DNase, LUCIGEN, cat No DM0715K), following the manufacturers instructions. After an incubation of 30 minutes at 37° C., they proceed to isolate the EVs according to the DMB methodology described in this invention.

Condition 4: The inventors started from 250 μl of plasma and 10,000 copies of the AKT p.E17K mutation (gBlock) to which they added 250 μl of reaction buffer (RB) and 25 μL of nuclease free water (NFW). After an incubation of 30 minutes at 37° C., they proceed to isolate the EVs according to the DMB methodology described in this invention.

| | | QUBIT | | ddPCR (number of Positive Events) | | | | |
|---|---|---|---|---|---|---|---|---|
| | Elution | (ng | TAPESTATION | KRAS | KRAS | AKT1 | AKT1 | Volume |
| CONDITION | volume | totals) | (Mean bp) | p.G12V | WT | p.E17K | WT | Loaded |
| 1 | 100 | 209 | 225 | 2222 | 3516 | 0 | 6493 | 32.4 |
| 2 | 100 | 249 | 229 | 2676 | 3978 | 0 | 5028 | 32.4 |
| 3 | 100 | 189 | 219 | 2170 | 3346 | 156 | 4945 | 32.4 |
| 4 | 100 | 200 | 216 | 3018 | 4425 | 371 | 5516 | 32.4 |

Table 4 shows the objectives and results obtained from the analysis of every conditions described above.

Together these results show that DMB co-precipitates approximately 15% of cfDNA, which is reduced by 50% when treated with DNase, and most of the obtained DNA seems to come from extracellular vesicles and not from free DNA.

### Example 15—EVs-DNA Isolated Using DMB is Suitable for Whole Exome Sequencing

Whole-genome sequencing of EVs-DNA obtained from 500 μL of plasma of 3 endometrial cancer patients using DMB technology was performed using Nimblegen SeqCap EZ MedExome (Roche) for library preparation and Illumina technology for the sequencing (Miniseq System, Illumina). For all samples a total of 37M of reads were obtained (14M, 8M and 13M for each sample), evidencing a good performance of the sequencing strategy.

TABLE 5
The quality of the samples was first tested using Qubit and
Tapestation High Sensitivity D1000 ScreenTape.

| ID | Qubit HS ng/μL | Total quantity (ng) | Test result |
|---|---|---|---|
| 19ID00822 | 2.22 | 133.20 | Qualified |
| 19ID00823 | 0.83 | 20.78 | Qualified |
| 19ID00824 | 0.80 | 20.05 | Qualified |

After library preparation, samples showed proper concentrations and integrity as show in Table 5 (TapeStation High Sensitivity D1000 ScreenTape).

After sequencing, FastQC and Quality control aligment showed a mean coverage of 27.25, 16.2 and 26.88 for each sample and a percent of target bases with coverage 30× of 8%, 1% and 6%, respectively, for each sample. This coverage is really good taking into account that the theoretic coverage for each sample was 3.5× for an initial estimation of 4.5M reads per sample with a panel of 47 Mb. The average sequence length was 146, 147 and 148 pb respectively.

TABLE 6
FastQC and Quality control alignment data.

| | Target Bases 30X | |
|---|---|---|
| ID NIM | (%) | Mean Coverage |
| 19\|000822 | 8 | 27,25 |
| 19\|000823 | 1 | 16,20 |
| 19\|000824 | 6 | 26,88 |

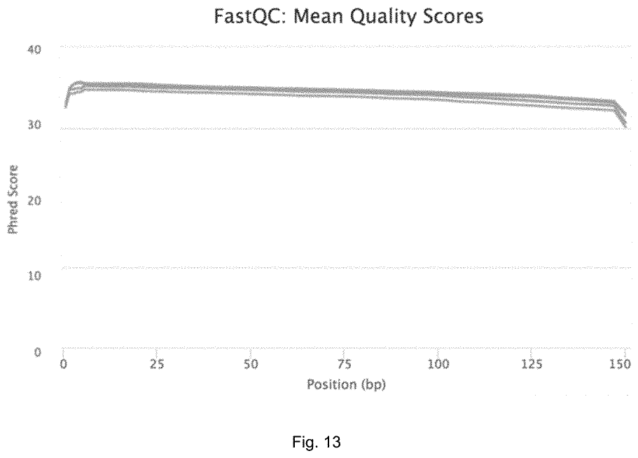

On the other side, the three samples analyzed in the study showed good quality scores as shown in FIG. 13.

### Example 16—MSI and CNV Analysis in Plasma EVs Isolated by DMB

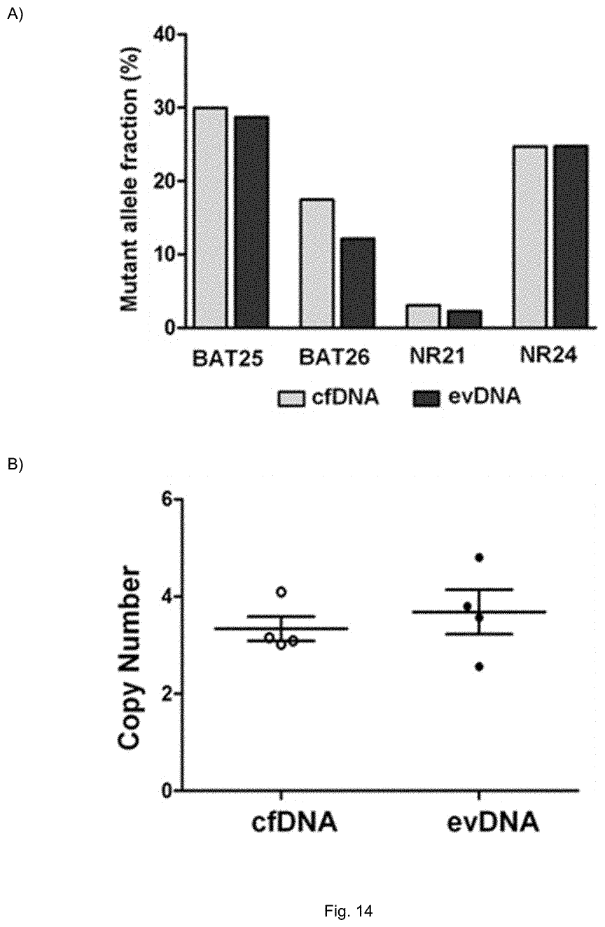

Microsatellite Instability (MSI) was analyzed by ddPCR in cfDNA from 5 ml of plasma or in the evDNA purified from the EVs isolated with DMB from 500 μl of plasma (FIG. 14A). It was also analyzed the measurement of MET copy number using ddPCR in cfDNA from 3 ml of plasma or in the evDNA purified from the EVs isolated with DMB from 500 μl of plasma (FIG. 14B).

### Example 17—Methylation Analysis in Culture Medium and Plasma EVs Isolated by DMB

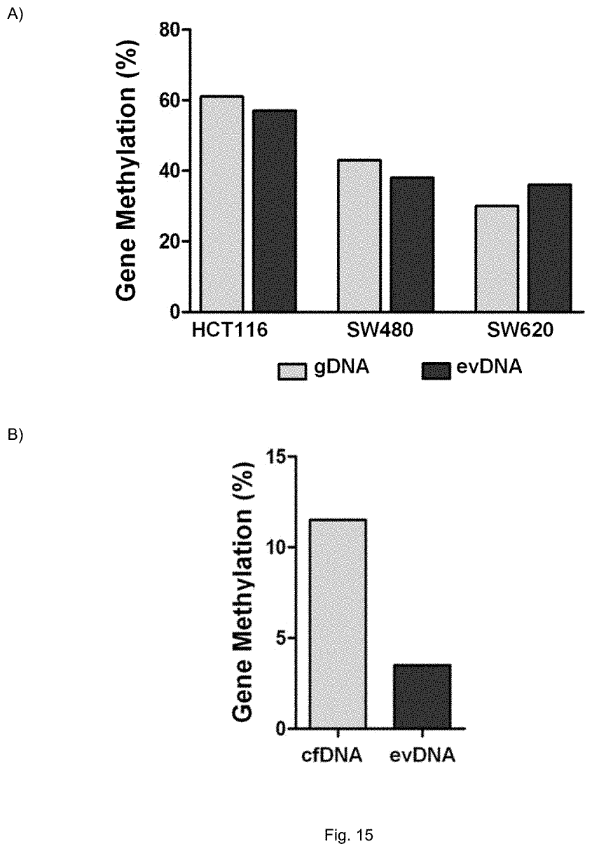

ddPCR analysis of the genomic DNA (gDNA) of different colorectal cancer cell lines (HCT116, SW480 and SW620) shows that are methylated at the targeted gene, as well as their EVs isolated using DMB, from 2 ml of culture medium (FIG. 15A). It was also analyzed gene methylation by ddPCR in cfDNA from 3 ml of plasma or in the evDNA purified from the EVs isolated with DMB from 500 μl of plasma, as observed in FIG. 15B.

### Example 18—mRNA Analysis in Plasma and Urine EVs Purified by DMB

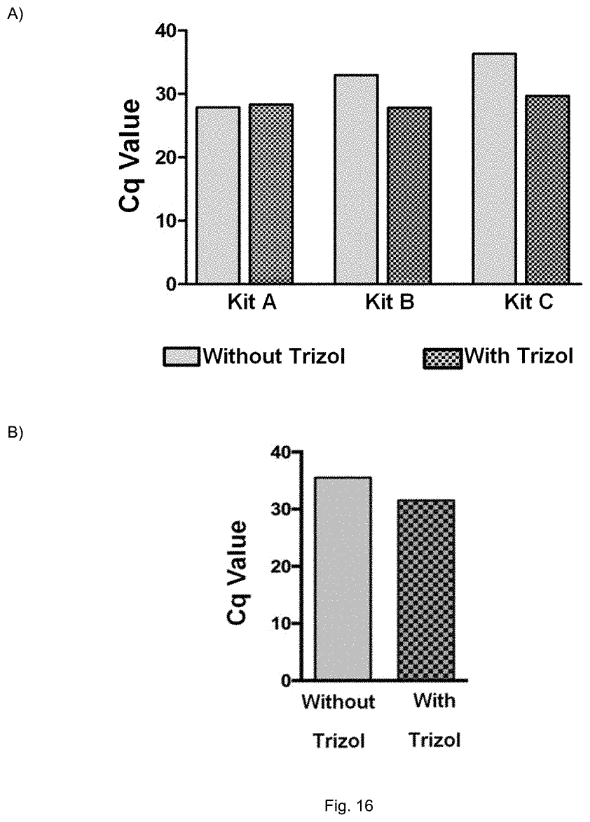

EV-mRNA purified after EVs isolation using DMB, from 3 ml of plasma samples (FIG. 16A) and 3 ml of urine (FIG. 16B) yields enough mRNA quantity to perform qPCR analysis. In some purification kits, a previous lysis step with Trizol improves performance.

1-15. (canceled)

16. An in vitro method for isolating nucleic acids associated to or contained inside extracellular vesicles (EVs) from a sample which comprises:
a) contacting the sample with the dimethylmethylene blue (DMB) dye at a pH comprised between 2 and 6.9;
b) incubating the mixture from a) at a temperature comprised between 0° C. and 40° C. for the time required for the formation of a DMB-EVs precipitate;
c) recovering the DMB-EVs precipitate; and
d) isolating the nucleic acids present in the precipitate.

17. The method according to claim 16, wherein the DMB is 1,9-Dimethyl-Methylene Blue zinc chloride double salt, and/or wherein step a) is performed at a pH between 3.3 and 3.6 and/or wherein step b) is performed at 4° C.

18. The method according to claim 16, wherein step a) is performed without previously isolating EVS from the sample.

19. The method according to claim 16, wherein step c) is performed by centrifugation.

20. The method according to claim 16, wherein the sample is a liquid biopsy or a tissue sample.

21. The method according to claim 16, wherein the nucleic acid is DNA or RNA.

22. A method selected from the group consisting of:
a) a method for diagnosing a disease or for determining the susceptibility of a subject to a disease comprising isolating nucleic acids according to the method of claim 16;
b) a method for determining the prognosis or for monitoring the progression of a disease in a subject comprising isolating nucleic acids according to the method of claim 16;
c) a method for monitoring the effect of a therapy for the treatment of a disease in a subject comprising isolating nucleic acids according to the method of claim 16;
d) a method for identifying compounds suitable for the treatment of a disease comprising isolating nucleic acids according to the method of claim 16; and
e) a method for designing a personalized therapy in a subject or for selecting a patient susceptible to being treated with a therapy for the prevention and/or treatment of a disease in a subject comprising isolating nucleic acids according to the method of claim 16.

23. The method according to claim 22, wherein the disease is cancer.

24. The method according to claim 22, wherein the method comprises analyzing the isolated nucleic acids to determine a genetic alteration of DNA or RNA.

25. A kit comprising dimethylmethylene blue (DMB) and a reagent capable of isolating nucleic acids from EVs.

26. A method for isolating nucleic acids associated to or contained inside EVs comprising the use of a kit comprising DMB or the use of a kit according to claim 25.

27. The method according to claim 20, wherein the liquid biopsy sample is one of serum, plasma, urine, saliva, synovial fluid, cerebrospinal fluid or semen.
